(19) United States
(12) Patent Application Publication
(10) Pub. No.: US 2007/0073555 A1
(43) Pub. Date: Mar. 29, 2007

(54) System and process for facilitating the provision of health care

(75) Inventor: Michael David Buist, Malvern, Victoria (AU)

Correspondence Address: MCKEE, VOORHEES & SEASE, P.L.C., 801 GRAND AVENUE, SUITE 3200, DES MOINES, IA, 50309-2721, US

(73) Assignee: PATIENTRACK PTY LTD., Armadale (AU)

(21) Appl. No.: 10/595,611

(22) PCT Filed: Oct. 29, 2004
(86) PCT No.: PCT/AU04/01499

§ 371 Date: Jun. 15, 2006

(30) Foreign Application Priority Data
Oct. 29, 2003 (AU) 2003905954

(51) Int. Cl. G06Q 10/00; A61B 5/00

(52) U.S. Cl. 705/2; 600/300

(57) ABSTRACT

A system for facilitating the provision of health care to patients, including computerised means (135) for logging patient data relating to health of the patients, and an administration system (105) in communication with the computerised means and configured to determine a risk status of each patient based on the patient data The administration system is also configured to, for each patient: transmit a first direction to a first health care provider to attend the patient, depending on the risk status of the patient; determine whether the first health care provider has confirmed attendance at the patient within a first time period; and transmit a second direction to a second health care provider to attend the patient within a second time period if attendance by the first health care provider was not confirmed.

### FIELD OF THE INVENTION

The present invention relates to a patient care system and process for facilitating the provision of health care to one or more patients.

### BACKGROUND OF THE INVENTION

Hospital ward environments have traditionally been operated on the basis of set procedures to be followed by health care personnel in relation to the provision of patient care. These procedures are often purely manual and rely greatly on the exercise of the skill and judgement of the attending health care personnel to ensure that the patient's needs are adequately attended to. Due to the significant reliance on the human faculties of the health care personnel, mistakes and oversights are inevitable in a busy hospital environment.

Statistics indicate that between 4% and 18% of hospital admissions have been associated with an adverse event caused by inadequate medical management. A recent study of the quality of Australian health care found that 16.6% of hospital admissions were associated with an adverse event, and that 18.5% of these adverse events resulted in permanent disability or death. In Australia, this translates to 14-18,000 deaths per annum and in the order of 50,000 injuries as a result of adverse events. The projected cost of these adverse events to the Australian healthcare system is in the order of AU$2 billion. Similar studies in the United States, United Kingdom and New Zealand have conformed the magnitude of this problem. Significantly, a further analysis of these events found that up to 70% of them were at least potentially preventable.

Many adverse events are caused by human error and failure of administrative processes. These may include:
(a) failure to synthesise, decide and/or act on available information;
(b) failure to request or arrange an investigation, procedure or consultation;
(c) lack of care or attention;
(d) failure to attend;
(e) delay; and
(f) misapplication of, or failure to apply, a rule, or use of a bad or inadequate rule.

It is desired to provide a system and process for facilitating the provision of health care to one or more patients, and a patient care process and system that alleviate one or more of the difficulties of the prior art, or at least provide a useful alternative.

### SUMMARY OF THE INVENTION

In accordance with the present invention, there is provided a process executed by a computer system for facilitating the provision of health care to one or more patients, including the steps of:
receiving patient data relating to the health of a patient;
processing said patient data to determine a risk status providing an indication of risk to the patient's health;
selecting a health care provider to attend said patient on the basis of said risk status; and
transmitting a direction to said health care provider to attend the patient.

The present invention also provides a system for facilitating the provision of health care to one or more patients, including:
computerised means for logging patient data relating to health of said one or more patients;
an administration system in communication with said computerised means and configured to determine a risk status of each of said one or more patients based on the patient data, said administration system being further configured to, for each patient: transmit a first direction to a first health care provider to attend the patient depending on the risk status of the patient; determine whether the first health care provider has confirmed attendance at the patient within a first time period; and transmit a second direction to a second health care provider to attend the patient within a second time period if attendance by the first health care provider was not confirmed.

Preferably, the second time period is equal to or less than the first time period.

Preferably, the first and second directions are effected by automatic transmission of a message to a portable electronic device associated with the respective first or second health care providers.

Preferably, the first and second directions are transmitted as wireless communications.

Preferably, the patient data includes data relating to a plurality of health parameters.

Preferably, the first direction is only transmitted when the risk status is equal to or above a threshold level.

Preferably, the first and second directions include information concerning the determined risk status of the at least one patient.

Preferably, the first and second directions include a request to confirm that the relevant health care personnel intends to comply with the direction.

Preferably, the administration system increases the risk status of the at least one patient if it determines that the first health care provider has not confirmed attendance at the patient within the first time period.

Preferably, the administration system is further configured to determine whether the second health care provider has confirmed attendance at the patient within the second time period and to transit a third direction to a third health care provider to attend the patient within a third time period if attendance by the second predetermined health care provider was not confirmed within the second time period.

Preferably, the third time period is equal to or less than the second time period.

Thus, if the patient is still not attended to by the first or second health care providers within a particular period of time, a third health care provider can be contacted to attend the patient. In effect, this allows the escalation of the risk status of the patient so that more senior medical staff can be contacted and shorter time frames may be provided for attending to the patient. Thus, the administration system can continue to monitor the patient's status and whether she has been attended to by the relevant health care personnel and can continue to transmit directions to health care personnel as appropriate. Thus, it is possible that four or five directions may issue and the risk status may be increased with the issue of each direction to ensure that the patient receives the necessary care.

Preferably, the computerised means include a plurality of computerised devices networked with, but located remotely from, the administration system.

Preferably, each computerised communication device is located nearby the at least one patient.

Preferably, the computerised device is a wireless handheld device.

Alternatively, the computerised device may be a personal computer with appropriate input means for logging the patient data.

Preferably, the administration system includes a centralised server having a risk assessment module for determining the risk status and a communications module for transmitting directions to health care personnel.

Preferably, directions to health care personnel are transmitted to the health care personnel by at least two contact devices. For example, the direction may be transmitted to a doctor's pager and, shortly thereafter, or simultaneously, be transmitted to the doctor's mobile phone. The direction may also be in the form of a recorded voice message directed to the doctor's office telephone number. If the patient is at the highest risk status, the

communication module may transmit the direction to all contact devices associated with the health care personnel at the same time.

The present invention also provides a process executed by a computer system for facilitating the provision of health care to a patient, including the steps of:
receiving patient data relating to the health of said patient;
determining a risk status of said patient based on said patient data;
transmitting a first direction to a first health care provider to attend the patient, the first direction including the risk status of the patient;
determining whether the first health care provider confirms attendance at the patient; and
transmitting a second direction to a second health care provider to attend the patient if attendance by the first health care provider was not confirmed.

Preferably, the second direction includes an increased risk status of the patient.

Preferably, the second direction includes a second predetermined time period for attending the patient.

Preferably, a first time period is associated with the determined risk status and the second time period is associated with the increased risk status.

Preferably, the second time period is equal to or less than the first time period.

Preferably, the method further includes the steps of:
determining whether the health care provider confirms attendance at the patient within the second time period; and
transmitting a third direction to a third health care provider to attend the patient if attendance by the second health care provider was not confirmed within the second time period. Preferably, the third direction includes a further increased risk status of the patient.

Preferably, the third direction includes a third time period for attending to the patient, the third time period being equal to or less than the second time period.

Preferably, the third time period is less than the first time period.

The present invention also provides a patient care system including:
at least one electronic device for recording patient data relating to the health of one or more patients;
a central server in communication with the at least one electronic device and configured to repeatedly contact one or more health care providers to request attendance at least one of the one or more patients if the patient data indicates that the health of the at least one patient is above a predetermined risk level and the at least one patient is unattended.

The present invention also provides a patient care system, comprising:
means for determining a risk level of a patient; and
means for repeatedly contacting one or more medical or health care personnel to attend the patient if the risk level is above a predetermined level and the patient is unattended.

The present invention also provides a patient care process executed by a computer system, including the steps of:
determining a risk level of a patient; and
repeatedly requesting one or more medical or health care providers to attend the patient if the risk level is above a predetermined level and the patient is unattended by said health care providers.

Embodiments of the invention provide systems concerned with the health of the individual patient by providing the bedside nurse and front line doctors with a real time solution for any deterioration in a patient's clinical status. The systems communicate with caregivers by graded alerts that are configurable to any healthcare setting. The graded alerts assist in task prioritisation for bedside nursing and medical staff based on the severity of the documented bedside observations. Advantageously, information capture by the systems allows the audit and analysis of individual patient and provider performance by any healthcare organisation.

### BRIEF DESCRIPTION OF THE DRAWINGS

Preferred embodiments of the present invention are hereinafter described, by way of example only, with reference to the accompanying drawings, wherein:

FIG. 1 is a block diagram of a preferred embodiment of a health care system;

FIG. 2 is a simplified flow diagram of a health care process of the system;

FIG. 3 is a flow diagram of a risk status process of the health care process;

FIG. 4 is a flow diagram of a neurological risk assessment process of the risk status process;

FIG. 5 is a flow diagram of a respiratory risk assessment process of the risk status process;

FIG. 6 is a flow diagram of a cardiovascular risk assessment process of the risk status process;

FIG. 7 is a flow diagram of a urinary risk assessment process of the risk status process;

FIG. 8 is a flow diagram of patient temperature risk assessment process of the risk status process;

FIG. 9 is a flow diagram of a communications process of the health care process; and

FIG. 10 is a block diagram of an alternative preferred embodiment of a health care system.

### DETAILED DESCRIPTION OF THE PREFERRED EMBODIMENTS

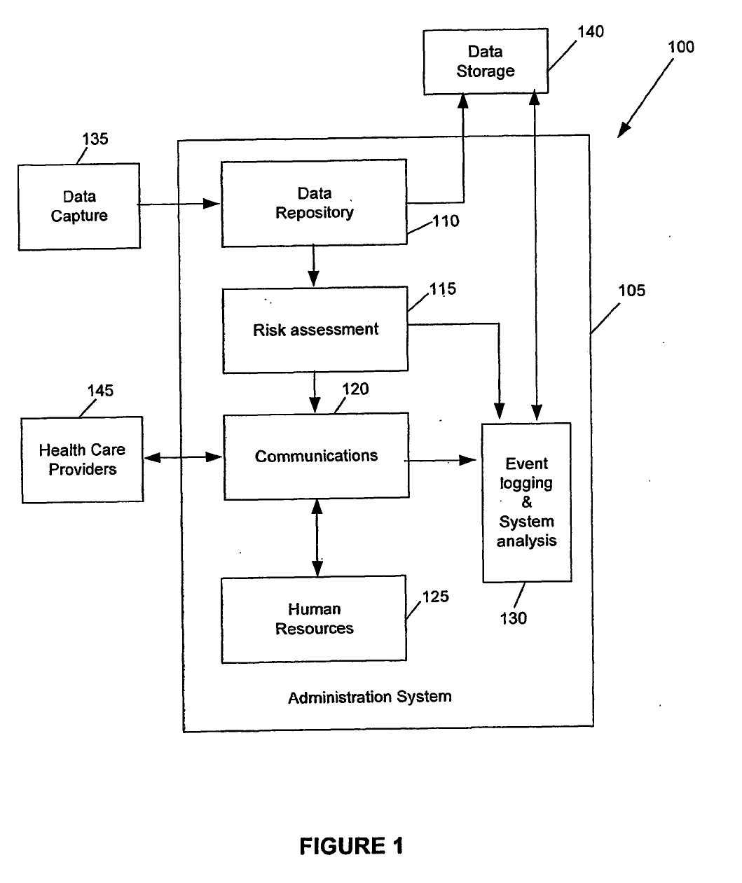

Referring now to FIG. 1, a health care system 100 provides a health care process that facilitates the provision of health care to one or more patients in a hospital ward environment. The health care system 100 includes an administration system 105 in communication with a number of remote data capture devices 135 for receiving clinical data for patients receiving care in the hospital. These data capture devices 135 are used by nursing or other clinical staff who examine each patient on a regular basis and input the clinical data, which includes measured health parameters (e.g., blood pressure, temperature, etc.) and other observations concerning the state of the patient's physical and/or mental health into the data capture device 135 for communication to the administration system 105. The administration system 105 processes the patient clinical data received in this way and communicates with one or more health care providers 145 (e.g., doctors, nurses, or a cardiac arrest team) as appropriate, depending on the severity of the patient's health status.

The administration system 105 is typically located on the hospital premises, but can alternatively be located remotely therefrom but in communication therewith. Patient clinical data collected from the data capture devices 135 are stored in a database of data storage 140 associated with the administration system 105.

The administration system 105 includes a data repository 110 for interfacing with data capture devices 135 and data storage 140. This data repository 110 provides the received patient clinical data to a risk assessment module 115 that processes the clinical data to determine a risk status of the patient. The risk status is provided to a communications module 120 within the administration system 105, which then transmits a direction or request (also referred to as an alert message) to one or more appropriate health care providers 145, requesting that they attend the patient, if the patient's risk status indicates this is required. The communications module 120 interfaces with a human resources module 125 within the administration system 105 to determine which individual health care providers to contact for a particular patient, as described below.

The administration system 105 also includes an event logging and system analysis module 130 for logging and tracking the performance of the administration system 105 and the health care provided by the hospital. The event logging and system analysis module 130 performs data mining, report generation, and critical analysis of the level of service being provided to patients. The event logging and system analysis module 130 enables hospital management to track clinical performance from both patient care and business efficiency perspectives. It enables management to identify issues that require resolution when the level of care provided does not meet expectations, and assists in identifying personnel performance requiring improvement.

In the described embodiment, the administration system 105 is a standard computer system, such as an Intel Architecture IA-32 computer, and the health care process is implemented as software modules, being the modules 110 to 130 stored on non-volatile (e.g., hard disk) storage associated with the computer system. However, it will be apparent that at least parts of the health care process can alternatively be implemented by dedicated hardware components, such as application-specific integrated circuits (ASICs). The data capture devices 135 preferably include portable or handheld computing or communications devices having wireless network interfaces, such as personal data assistants (PDAs), mobile telephones incorporating PDA functions, notebook or tablet personal computers, but may also or alternatively include standard desktop computers or other computing devices with non-wireless network interfaces.

The system 100 is particularly well suited to a general ward environment where the potential for adverse health care events and failure to detect such events is high. The system 100 receives and processes patient clinical data of the type traditionally recorded by nurses in hand-written charts. With the system 100, nurses input patient clinical data at regular intervals directly into one of the data capture devices 135 instead of the traditional hand-written bedside observation chart. The patient clinical data is then transmitted in electronic form from the capture device 135 to the data repository 110, after which it is forwarded to the risk assessment module 115 and data storage 140.

The risk assessment module 115 continuously evaluates any available parameters of the patient clinical data (including blood pressure, heart rate, respiration rate, oxygen saturation, consciousness level, urine output, temperature, and pain score) against predetermined safety benchmarks for each parameter stored in the data repository 110. The risk assessment module 115 assigns a risk status that represents the total health risk to the patient based on all of the available clinical data for that patient by comparing the received values for each parameter with a predetermined set or range of values for that parameter that are considered to define normal or acceptable values for a patient in reasonable health. The risk status is assigned as one of six levels, from level 0 to level 5, in order of increasing severity. The higher the risk status level, the more likely that a patient may be experiencing, or may be about to experience, a critical or otherwise adverse health event. Consequently, the risk status determines whether an intervention activity is required. Each hospital sets its intervention activities according to its own risk criteria. When a patient's risk status level is equal to or higher than a predetermined intervention threshold value, the communication module 120 communicates a message to an appropriate nursing or physician resource 145 requesting appropriate intervention action, such as bedside attendance or cardiac arrest management. The higher the level of risk, the shorter the response time allowed, and the more senior the health care provider(s) requested to attend the patient.

The communications module 120 that delivers the medical intervention alerts to health care personnel 145 is configurable to enable multiple communications devices (e.g., mobile phone/SMS, pagers, PDA, etc.) to be used to contact each doctor or other health care provider 145. Each alert includes a short message requesting the health care provider to confirm that they intend to attend the patient, and also provides the patient's clinical data, the patient's risk status, and the associated required response time. If the health care provider does not respond to an alert within a configurable acceptance time period, the communication module 120 identifies the next most appropriate health care provider and sends a request to that person to undertake the relevant intervention activity. If that person also does not respond within the acceptance period, or responds but does not attend the bedside within the required response time, the communication module 120 contacts the next most appropriate health care provider, and so on. The administration system 105 allows the hospital to configure the required acceptance and response times for each risk status level in accordance with hospital policy. Hospital administration is alerted by the administration system 105 to situations where there are health care resource problems (quality and quantity), particularly in emergency circumstances that require administrative intervention.

The human resources module 125 maintains current staffing and roster records and supports the communications module 120 by providing up-to-date information on the health care providers that are available to respond to requests for bedside intervention. This prevents intervention requests being made that are not likely to be met due to unavailability of health care personnel.

The administration system 105 can be configured by the hospital to set operational parameters such as the kind and frequency of data capture, the basis upon which risk assessment is performed, the acceptance and response times allowed for responding to alert messages, and the communication methods or devices used to send them. The administration system 105 also provides a user interface to the system 100 so that health care personnel and hospital administrators can review patient clinical data and performance data to ensure that an appropriate level of care is being provided to patients.

Other administration functions also provided by the administration system 105 include:
(i) User rights and privileges;
(ii) Graphical user interface configuration and content management;
(iii) Initial and ongoing systems configuration;
(iv) Systems settings, backup and management;
(v) Data import and extraction;
(vi) Updating, refining and logging risk assessment benchmark changes; and
(vii) Privacy requirements.

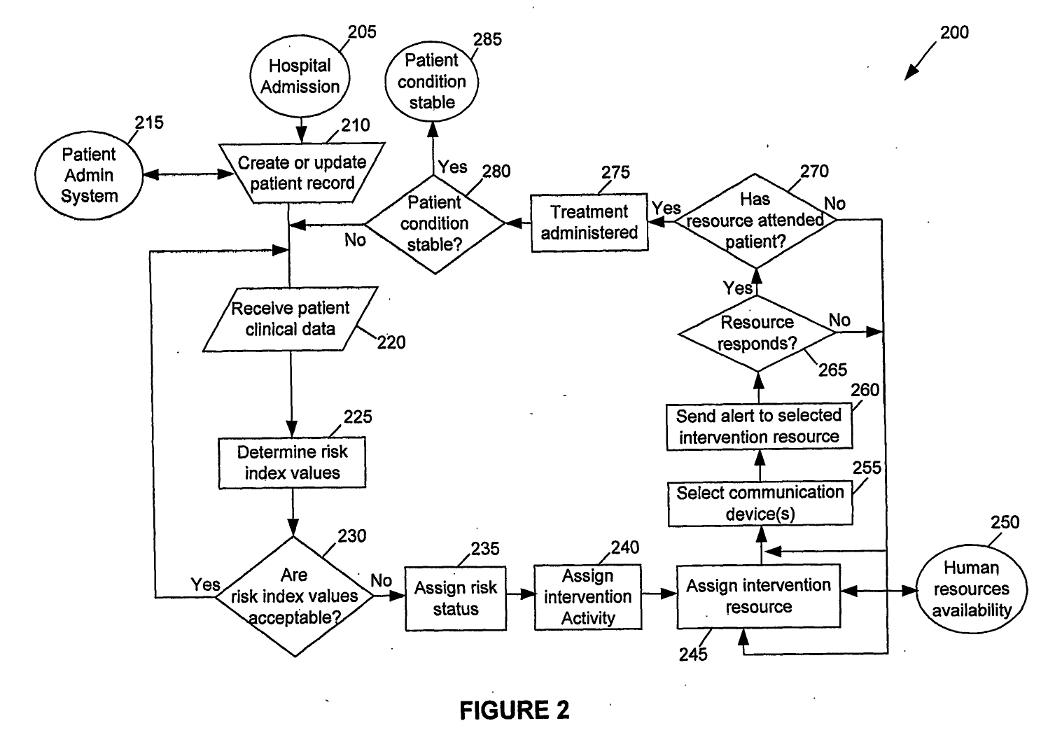

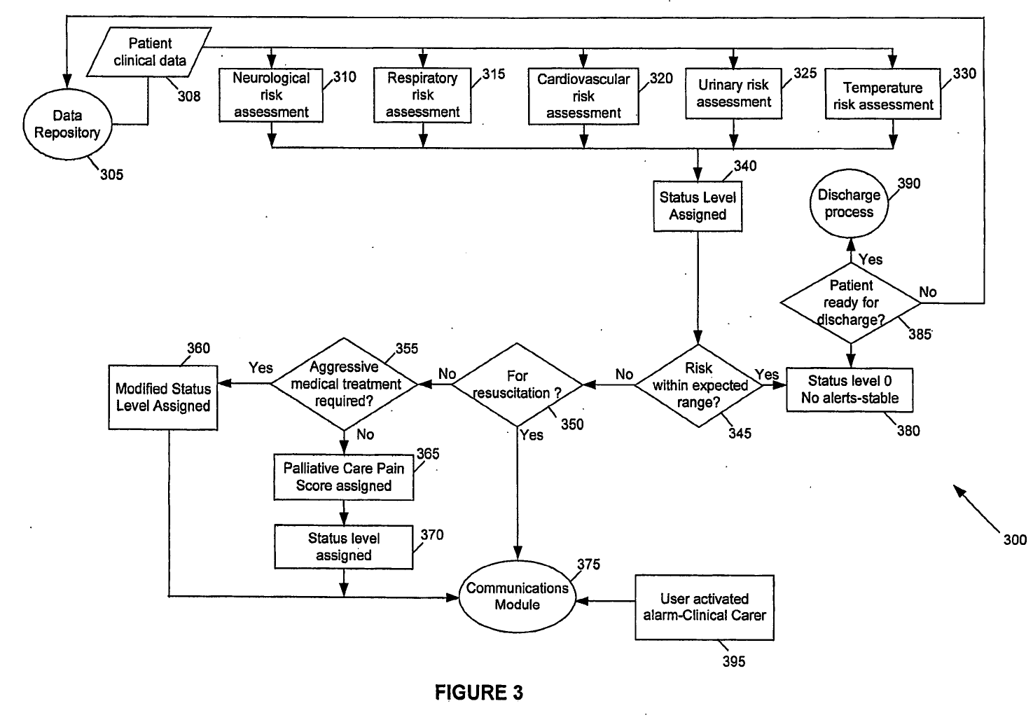

Referring now to FIG. 2, the health care process 200 of the health care system 100 is described. The flow diagram shown in FIG. 2 provides a summary or overview of the major steps of the health care process of the health care system. However, it should be understood that the complete health care process includes additional steps that are not shown in FIG. 2, but that are described below. More detailed flow diagrams of sub-processes that are each part of the complete health care process are shown in FIGS. 3 to 9, and are described in detail below. Returning to FIG. 2, the health care process 200 can be considered to begin with admission of a patient to the hospital at step 205. Following admission, a patient record is created at step 210 by a nurse or an administration staff member. If the patient has not previously been a client of the hospital, a patient profile providing pertinent personal and clinical data is created. Otherwise, if the patient is an existing client of the hospital, the nurse need only establish a new record for that patient to correspond with the patient's current health complaint. In order to establish the patient record and/or profile at step 210, the nurse may query the administration system 105 at step 215.

The patient profile includes the following patient information:
(i) Patient details (name, date of birth, treatment address, patient identification, ward, etc.) with appropriate privacy security. This information establishes a unique patient record so that a patient's risk status can be monitored.
(ii) Patient clinical data, including bedside observation parameters that form the basis of risk assessment, including blood pressure, heart rate, respiratory rate, level of consciousness, temperature, pain score, oxygen saturation, urine output, Do-Not-Resuscitate (DNR) status, and may include other parameters such as co-morbidity factors. The system 100 can be configured to store and process additional observation parameters if they are considered to play an important role in indicating the possibility of an adverse health event.

Data is entered by authorised medical staff including nurses, junior doctors, registrars and consultants by entering a login identifier (e.g., staff number) and a corresponding password.

The system 100 can be configured to request medical staff to collect data at selected time intervals (e.g., every 15, 30, or 60 minutes), depending on the patient's risk status and hospital policy.

The system 100 follows a thin client model, with the patient information held on a central database 140. The patient record, including patient clinical data, can be displayed on the data capture device 135 at the bedside if requested by authorised personnel. For example, the capture device 135 can be configured to read a barcode on an ID tag of a health care provider, preferably in conjunction with a corresponding password, to determine the relevant authorisation. Nursing staff can use the administration system 105 to print hard copies of observation charts and other patient data via the wireless network to any one of a number of network printers throughout the hospital. The data repository 110 can be configured to retrieve data from existing hospital systems, such as a PAS (patient administration system), if necessary.

Once a patient record has been established for the patient at step 210, the patient undergoes clinical observations, and patient clinical data concerning the patient's health and physical/mental state is received at step 220. Traditionally, that data has been captured manually by a nurse or medical officer by writing it on a paper chart or other document. In contrast, the health care system 100 uses electronic data capture device 135 s (e.g. a PDA, PC, or tablet PC) whereby nurses input clinical data into the device 135 at or near the patient's bedside. The clinical data entered by hospital staff using capture device 135 is transmitted over the hospital's wireless network to the data repository 110, and is processed by the risk assessment module 115 at step 225 to generate patient risk index values corresponding to the various parameters (blood pressure, etc.) of the clinical data, as described below. If the patient's risk index values are found to be acceptable at step 230, the patient continues to undergo clinical observation at step 220. Otherwise, if the risk index values are outside acceptable values, indicating that the patient should be attended by health care personnel, a risk status is assigned at step 235. The assigned risk status can be directly determined by the generated risk index values or may additionally take into account personal or general patient characteristics such as age, weight, sex or past medical history. Once a risk status is assigned to the patient at step 235, a corresponding intervention activity is determined at step 240 according to the hospital's desired procedures.

An intervention resource (e.g., health care provider) is then assigned at step 245, depending on the determined intervention activity and the available human resources (determined by the human resources module 125 at step 250 ).

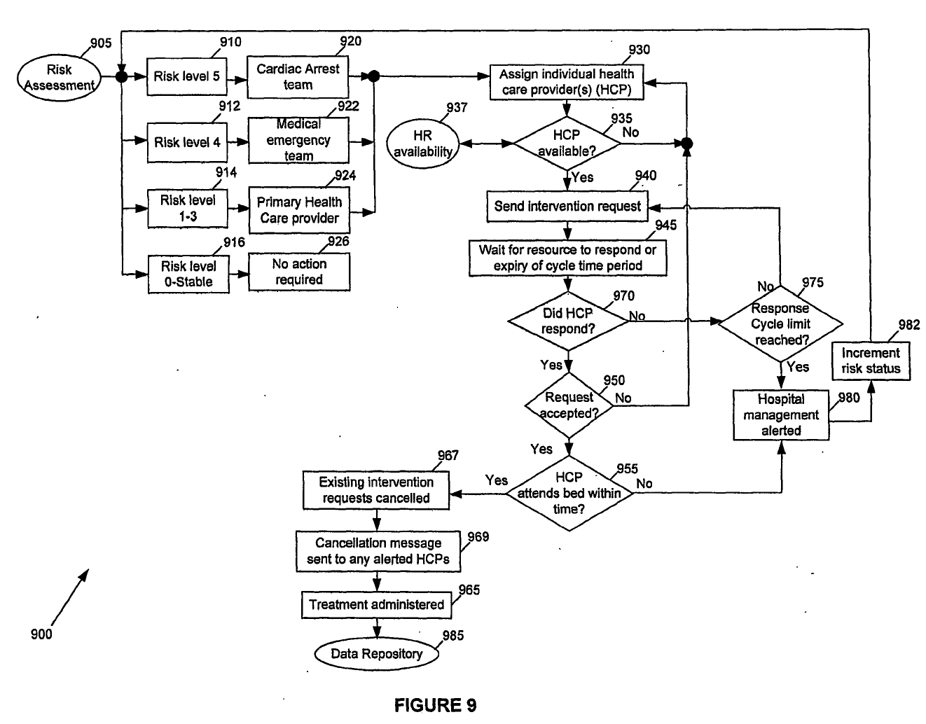

Once an appropriate health care provider has been identified and assigned at step 245, one or more communications devices associated with the assigned health care provider, for example a mobile phone, pager or personal digital assistant associated with the care provider, is selected at step 255 and an alert message is sent to the selected device at step 260. The alert message requests the health care provider's attendance to the patient, providing also the patient's risk status and requesting an affirmative response from the care provider. At step 265, if the health care provider responds within a displayed acceptance time period (using one or more of the methods or devices described below in relation to FIG. 9), and the health care provider attends the patient at step 270, treatment is administered to the patient at step 275. If the health care provider does not respond at step 265 to the request for intervention with the specified acceptance period, an alternative communications device associated with the care provider can be selected at step 255 and contacted at step 260, or an alternative health care provider can be assigned at step 245, depending on the risk status of the patient. This is also the case if a health care provider responds at step 265 but does not attend the patient within the required response time at step 270.

After treatment has been administered to the patient at step 275, the patient's condition is assessed at step 280 and, if considered to be unstable, the patient continues to undergo clinical observation at step 220. Otherwise, the patient's condition is considered to be stable at step 285 and the patient may or may not undergo further clinical observation at step 220, depending on hospital policy or the judgement of the attending health care provider.

Thus, as will be appreciated from the above description of the patient care process 200, each patient is monitored and appropriate health care providers alerted in an escalating manner to ensure that the patient is not overlooked or forgotten due to the unresponsiveness or unavailability of any one or more health care providers.

Various aspects of the health care process are described in more detail below, with reference to FIGS. 3 to 8 and corresponding subprocesses of the health care process. FIG. 3 shows a risk status process 300 executed by the risk assessment module 115, beginning with the reception of patient clinical data 306 from the data repository 110 at step 305. This received patient clinical data is classified into five categories corresponding to the following five key health systems observed for each patient:
System 1 —Neurological;
System 2 —Respiratory, respiration rate and oxygen saturation;
System 3 —Cardiovascular, blood pressure and heart rate;
System 4 —Urine System; and
System 5 —Temperature.

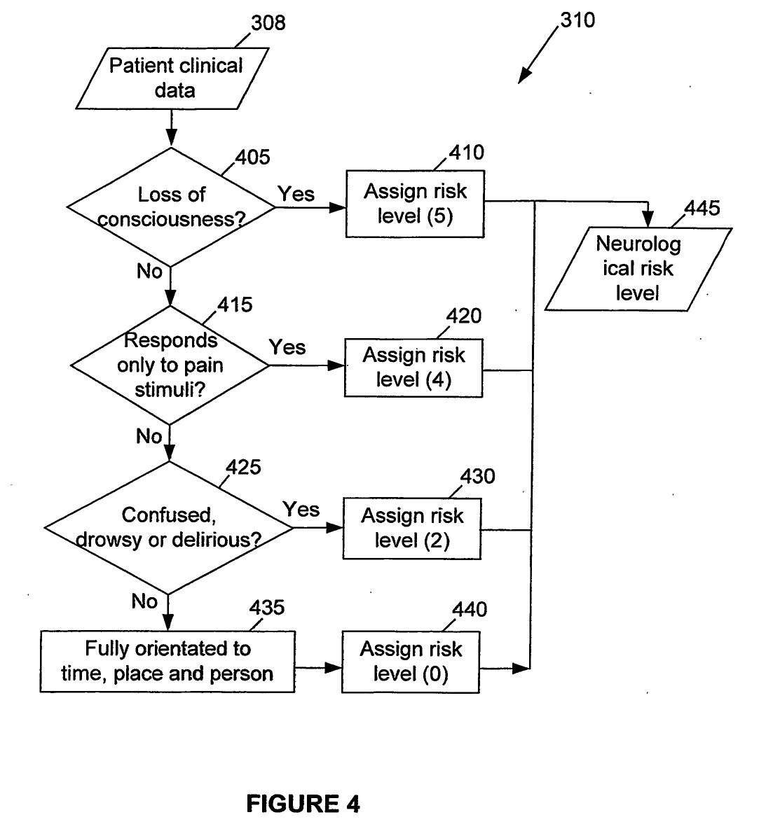

Patient health parameters corresponding to the neurological, respiratory, cardiovascular, urinary and temperature health systems are analysed concurrently by respective risk assessment processes 310, 315, 320, 325 and 330 described further below in relation to FIGS. 4 to 8. Each of these processes determines a risk index value in the range of 0 to 5 representing a health risk for the corresponding key health system.

Referring now to FIG. 4, a neurological risk assessment process 310 begins by determining the level of consciousness of the patient at step 405. If the patient has lost consciousness, a neurological risk index 445 is assigned at a value of 5 at step 410. If the patient is conscious, the patient's response to pain stimuli is observed at step 415. If the patient only responds to pain stimuli, the neurological risk index 445 is assigned a value of 4 at step 420. Otherwise, the patient is observed for confusion, drowsiness, delirium at step 425. If any such observations are present, the neurological risk index is assigned a value of 2 at step 430. Otherwise, if the patient's level of consciousness is such that he or she is fully aware and conscious and capable of appropriate mental function at step 435, a risk index of 0 is assigned at step 440. The neurological risk index 445 determined at step 405, .410, 420, 425, 430 or 400 is provided as output and the process ends.

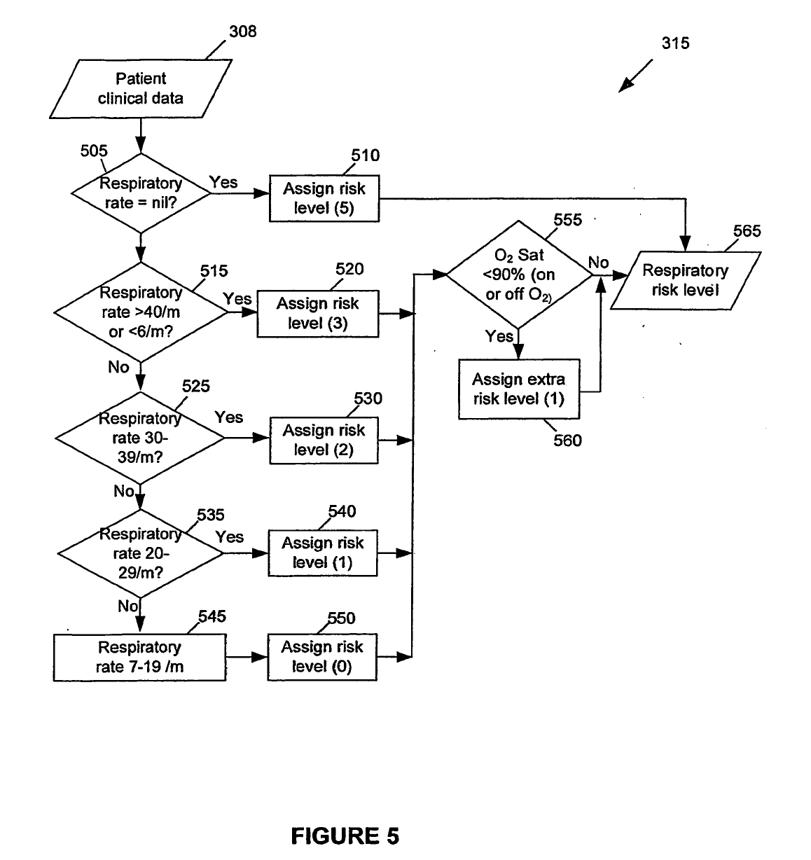

Referring now to FIG. 5, a respiratory analysis process 315 begins at step 505 by determining whether the patient's respiratory rate is nil. If so, then a risk index of 565 is assigned a value of 5 at step 510. Otherwise, if the respiratory rate is greater than 40 breaths per minute or less than 6 breaths per minute at 515, a respiratory risk index of 3 is assigned at step 520. Otherwise, if the respiratory rate is determined to be between 30 and 39 breaths per minute at step 525, a risk index of 2 is assigned at step 530. Otherwise, if the respiratory rate is between 20 and 29 breaths per minute at step 535, a risk index of 1 is assigned at step 540. Finally, if the patient's respiratory rate is between 7 and 19 breaths per minute at step 545, a risk index of 0 is assigned at step 550. The risk index thus determined provides a respiratory risk index 565. However, if a patient's oxygen saturation level is less than 90%, whether that patient is receiving extra oxygen or not, this is considered to be an indicator of greater risk and the respiratory risk index assigned at step 520, 530, 540 or 550 is increased by 1 at step 560. Otherwise, the respiratory risk assessment process 315 ends, providing the respiratory risk index 565 as output.

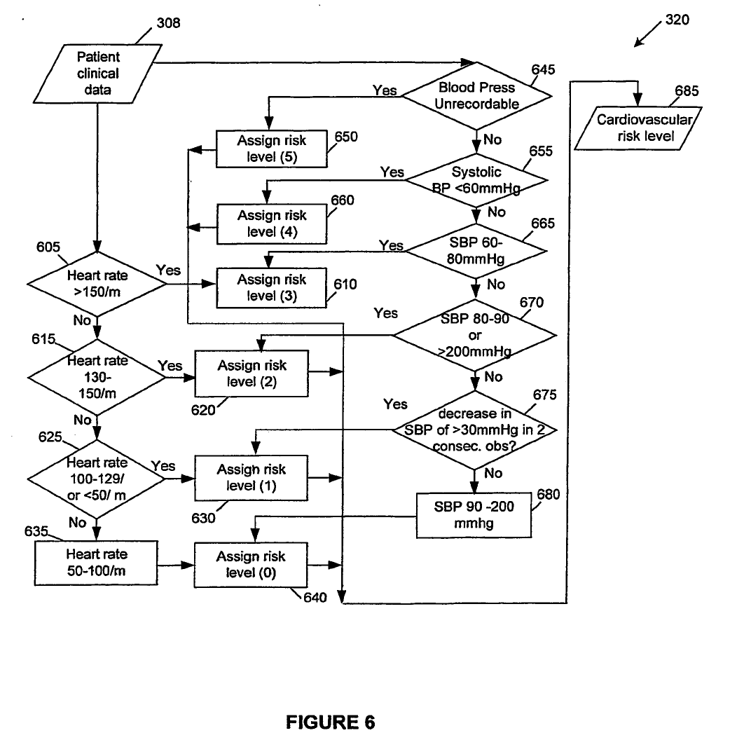

Referring now to FIG. 6, a cardiovascular risk assessment process 600 begins with an examination of the heart rate at step 605. If the patient's heart rate is determined to be greater than 150 beats per minute, a cardiovascular risk index of 685 is assigned a value of 3 at step 610. If the patient's heart rate is determined to be between 130 and 150 beats per minute at step 615, a cardiovascular risk index of 2 is assigned at step 620. If the patient's heart rate is determined to be between 100 and 129 beats per minute or less than 50 beats per minute at step 625, a cardiovascular risk index of 1 is assigned at 630. If the patient's heart rate is determined to be in the normal range of between 50 and 100 beats per minute at step 635, a cardiovascular risk index of 0 is assigned at step 640.

In addition to examining the patient's heart rate, the patient's blood pressure is examined at step 645, and if the patient's blood pressure is unrecordably low, a cardiovascular risk index of 5 is assigned at step 650. If the patient's systolic blood pressure is less than 60 mm of mercury at step 655, a cardiovascular risk index of 4 is assigned at step 660. If the patient's systolic blood pressure is between 60 and 80 mm of mercury at step 665, a cardiovascular risk index of 3 is assigned at step 610. If the patient's systolic blood pressure is between 80 and 90 or greater than 200 mm of mercury at step 670, a cardiovascular risk index of 2 is assigned at step 620. If there is a decrease in systolic blood pressure of greater than 30 mm of mercury in two consecutive observations at step 675, a cardiovascular risk index of 1 is assigned at step 630. If the patient's systolic blood pressure is between 90 and 200 mm of mercury at step 680, a cardiovascular risk index of 0 is assigned at step 640. The cardiovascular risk index 685 is provided as output.

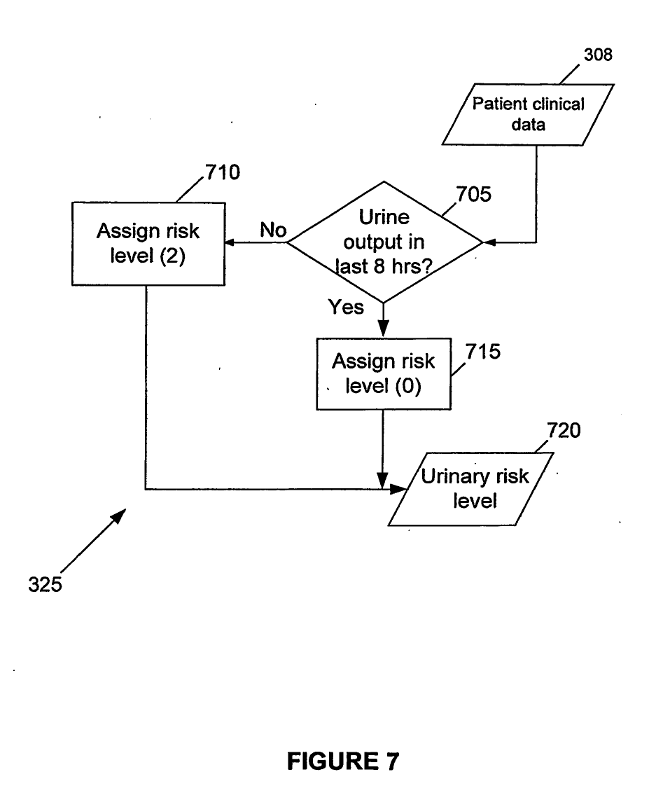

Referring now FIG. 7, a urinary risk assessment process 325 begins by determining the patient's urine output. If it is observed at step 705 that urine output of more than 400 mls occurred in the last eight hours, a risk index 720 is assigned a value of 0 at step 715. Otherwise, a urinary risk index of 2 is assigned at step 710. The urinary risk assessment process 325 then ends, providing the urinary risk index 720 as output.

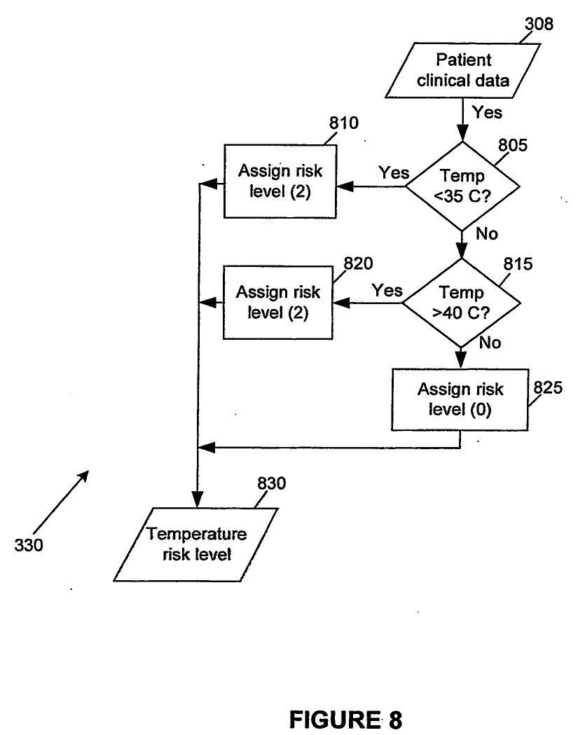

Referring now to FIG. 8, a temperature risk assessment process 330 begins by determining whether the patient's temperature is less than 35° C. at step 805, and, if so, a temperature risk index 830 is assigned a value of 2 at step 810. If the patient's temperature is greater than 40° C. at 815, a temperature risk index of 2 is assigned at step 820. Otherwise, if the patient's temperature is between 35 and 40 ° C., a temperature risk index of 0 is assigned at step 825. This ends the temperature risk assessment process 330.

Returning to FIG. 3, the neurological, respiratory, cardiovascular, urinary and temperature risk index values 445, 565, 685, 720 and 830 are used to assign a risk status to the patient at step . 335, as described below, representing the overall health risk for the patient.

The system 100 uses at least six risk status levels and associated intervention responses, as follows:

Status Level 0—Patient Stable (No Intervention or Attendance Required)

This status level indicates that the patient's clinical data parameters are within acceptable values and the patient's overall condition is considered to be stable.

Status Level 1—Non Urgent Review (Attendance required within 3-8hrs)

This indicates the patient's observations are outside expected range but no immediate or urgent intervention activity is required. The patient should be attended within about 3 to 8 hours. The implied instability may self-correct as the patient continues his or her recovery or may be an indicator that there is an issue that requires treatment. An issue requiring treatment would be indicated by a continued non-zero risk status or increased risk index values.

Status Level 2—Timely Review Required (Attendance Required Within 1-3hrs)

This status level indicates that the patient's observation values are abnormal and the patient requires review within 1-3 hours. The implied instability may self-correct as the patient continues his or her recovery or may be an indicator that there is an issue that requires addressing. An issue requiring addressing would be indicated by a continued non-zero risk status or an increased risk index value.

Status Level 3—Urgent Assessment Required (Attendance Required Within 10 mins-60 mins)

This status level is typically used when one or more risk index values are substantially outside acceptable values in one or more of the key health systems or when the risk status has been escalated from level 2. This indicates an elevated risk of a critical health event and indicates that the patient's health is unstable. The patient should be attended within about 10 to 60 minutes.

Status Level 4—Immediate Response Required (Attendance Required Within 0-10 mins)

This status level is assigned when the risk index values indicate that one of more of the patient's health parameters are substantially outside the expected range indicating that the patient is at risk of an impending cardiac arrest or other serious health risk. The patient should be attended within 10 minutes.

Status Level 5—Cardiac Arrest Call (Immediate Attendance Required)

This status level is assigned if the Patient is suffering a cardiac arrest or other life-threatening condition and requires immediate intervention by medical personnel, such as the cardiac arrest team.

A greater number of risk status levels and greater variety of intervention activities can be configured into the system 100 if desired to accommodate any perceived need for such. Similarly, the prescribed response times can be modified to accord with the desired service response level of the hospital or ward.

As described above, a patient's health risk associated with any one of the five health systems is assessed on a 0-5 point scale to provide neurological, respiratory, cardiovascular, urinary and temperature risk index values 445, 565, 685, 720 and 830, from which the risk status is assigned one of the six levels described above using a set of risk assessment rules that can be configured by the hospital's risk criteria. However, unless modified by the hospital, the risk status level of a patient is determined using the following rules:
1. If any of the risk indexes determined for the five health systems is equal to the maximum value (i.e., 5 points) then the patient has reached a critical threshold and immediate intervention is required. The patient is assigned risk status level 5.
2. Otherwise, if two or more of the risk index values are non-zero, then the highest risk index value is selected and incremented by 1 point. The resulting value provides the risk status level for the patient.
3. If an intervention request has been sent to an assigned health care provider and that person has not responded within the required time limit, the risk status is increased by 1 level over and above the risk status assigned to the patient on the basis of clinical observation data alone.
4. Notwithstanding the above, no more than 5 risk index points can be assigned to a patient, and a maximum of 4 points is assigned if the patient is not actually undergoing a cardiac arrest or similar level of danger to the patient's health (i.e., loss of consciousness).
5. Notwithstanding the above, if a not-for-resuscitation (NFR) order is in place for the patient, the risk index value for that patient is not allowed to exceed 4 points.

However, it will be apparent that the patient clinical data can be processed in a variety of alternative ways to generate a risk status. Generally, the larger the deviation from the acceptable range for each parameter, the higher the risk index and the more likely a patient will experience, or is currently experiencing, a critical health event. On the basis of the resulting risk, the risk assessment module 115 automatically assigns to the patient a risk status level selected from a set of predetermined risk status levels. Each risk status level is associated with a corresponding intervention response or activity. The risk status thus determines the appropriate intervention response, if any, which is communicated to the appropriate health care provider(s) by the communication module 120.

Each hospital sets the intervention activities for each risk status level (0-5) that is assigned on the basis of the risk index values and that meet the hospital's risk criteria. When a patient's risk index is higher than an intervention range threshold (e.g., level 0), the system 100 communicates a message to the appropriate health care provider 145 via the communications module 120.

The risk assessment module 115 continuously assesses the patient's risk status based on observation data until the patient is either discharged or dies.

The purpose of the NFR option is to ensure that patient's wishes are met in respect of treatment and to allow the terminally ill to receive appropriate palliative care. After a patient risk index is generated and a corresponding risk status assigned, the risk assessment module 115 checks the patient's NFR status. If the patient's NFR status allows resuscitation, then the communications process is executed in a standard manner. Otherwise, if the patient is designated NFR, the system 100 executes another decision process, as follows.

If a patient is designated NFR but requires aggressive medical treatment, the patient shall receive full treatment to aid recovery, other than resuscitation in the event of a cardiac arrest. Accordingly, the patient's risk status level can only ever be within the 0-4 range, as resuscitation is no longer an option, and the cardiac arrest team will not be called in the event of a cardiac arrest. The patient will, however, undergo aggressive treatment to assist in recovery.

A patient who is terminally ill and designated NFR is assessed outside the risk status process of FIG. 3, and only in reference to the following pain score. As resuscitation and aggressive treatment are not viable treatment options, the patient's risk status level is limited accordingly to a maximum of risk status level 3 according to the following table:

| Pain/Comfort Score | Risk Status Level |
|---|---|
| 0-2 | 0 |
| 3-4 | 1 |
| 5-7 | 2 |
| 8-10 | 3 |

The pain/comfort score is determined according to standard methods known to health care providers.

The risk status level and any corresponding intervention requirements for that risk status level are handled pursuant to normal operation of the communications module 120. The risk assessment rules described above do not apply to NFR/Palliative care patients. An NFR order can only be assigned or reassigned to a patient by the patient's primary health care provider or the physician's immediate manager.

In addition to the risk assessment rules described above (or as modified by the hospital), a nurse or physician can at any time manually upgrade a patient's risk status to any higher level using a “User Activated Alarm” feature 395 on the bedside data capture device 135. This feature may be used when it is obvious that a patient is experiencing an emergency situation. For example, the patient may have fallen out of bed and struck her head and is bleeding profusely. However, a user cannot manually downgrade a risk status or an intervention activity request.

Returning to FIG. 3, if the patient's risk status is within the acceptable range (e.g., risk status level 0) at step 345, the patient is considered to be stable at step 380. At step 385, the attending care provider determines whether the patient is ready for discharge and, if so, the patient is processed for discharge at step 390. Otherwise, the patient continues to undergo clinical observation.

If the assigned risk status indicates that the patient's health may be unstable, a check is made at step 350 as to whether the patient has a not-for-resuscitation RR) order in place. For most patients, this will not be the case and the process 300 will proceed to initiate communication with appropriate health care personnel via the communications module 120 at step 375. For those patients with an NFR order in place, there is a further check at step 355 as to whether aggressive medical treatment is required. If so, a modified risk index is assigned at step 360 as described above. If aggressive medical treatment is not required, a palliative care pain score is assigned at step 365, and a corresponding risk status is assigned at step 370, as described above. Once any modification to the patient's risk status is made at step 360 or step 370, appropriate health care personnel can be contacted at step 375.

After the risk assessment module 1 15 assesses risk to the patient's health based on clinical data for that patient and assigns a risk status control intervention activity, the communications module 120 executes a communications process 900, as shown in FIG. 9, deliver a corresponding alert message requesting the intervention activity and including patient information to the responsible physician, nurse, or other health care provider, and escalates the alert, if necessary.

The communications process 900 begins following receipt of the assigned risk status from the risk assessment module 115 at step 905. If the risk status is level 5 at step 910, a cardiac arrest team is assigned to the patient at step 920. In the sense employed in this specification, the term cardiac arrest should be understood to indicate any serious health risk, rather than being limited to a situation where the patient's heart has stopped. Accordingly, the term cardiac arrest is used herein in a broad sense to indicate any serious health condition requiring immediate intervention, such as when the patient has lost consciousness or has stopped breathing. If at step 912 it is determined that a risk status of level 4 was assigned to the patient, a medical emergency team 922, or other appropriate urgent response as determined by the hospital, is assigned to the patient. If at step 914 it is determined that a risk status of level 1 to 3 was assigned, a primary health care provider is assigned to the patient at step 924. If at step 916 it is determined a risk status of level 0 was assigned to the patient, it is considered that no further action is required at step 926. If a health care resource is required to attend the patient, (i.e., risk status >level 0), such a resource is assigned at step 930; that is, at least one individual health care provider of the type determined at step 920,922, or 924.

Risk status level 3-5 intervention activities may require differing levels of medical seniority and specialisation. For example, risk status level 5 requires cardiac arrest team intervention, whereas risk status level 3 may only require a junior doctor or ward nurse at step 924. The administration system enables hospital administrators to assign different types of health care personnel to requests for interventions, as shown in the Table below.

| Risk Status | | Response Resource |
|---|---|---|
| Level | Required Intervention Activity | Required |
| 0 | Patient data is within expected | NIL |
| | range and patient's overall | |
| | condition is described as stable | |
| 1 | Non Urgent Review (3-8 hrs); | Alert/Information |
| | Patient observations are | sent to patient primary |
| | moderately outside the expected | health care provider |
| | range | Nurse/junior physician |
| 2 | Timely review required (1-3 hrs); | Registrar level |
| | patient observations are | response |
| | materially outside expected | |
| | range | |
| 3 | Urgent Assessment required | Registrar level |
| | (10-60 minutes) | response |
| 4 | Immediate response required | Registrar/Consultant |
| | (0-10 minutes) | Medical Emergency |
| | | Team |
| | | Outreach |
| 5 | Cardiac arrest (immediate) | Cardiac arrest team |

The hospital can configure its individual risk profile and thus the types of resources that are selected to respond to the various risk status levels described above. For example, one hospital may choose to assign nurses to investigate risk status level 2 patients, whereas other hospitals may assign junior doctors.

Having assigned a risk status and a corresponding type of care provider, the system 100 can be configured to prioritise the actual individual human care providers that are asked to respond. For example, in the case of a risk status level 3 patient, the patient's primary health care provider may be selected at steps 924 and 930 to attend. If, at steps 970 and 975, it is determined that the primary health care provider has not responded by the expiry of the required acceptance period, the communications module 115 then determines the next most appropriate health care provider available as described below, and sends an appropriate alert message to that person. Health care providers can be defined by a number of categories or types, including peer group (speciality and sub-speciality), seniority, commercial grouping (e.g., private practice partners), current rostered resources, or other criteria associated with care providers. The human resource module 125 is checked at step 935 to ensure that only resources that are listed as available are selected as intervention resources. The intervention request can be sent at step 940 via any one or more of a variety of methods or devices, including the following:
1. As a Short Message Service (SMS) message automatically generated and sent to the required health care provider(s) (HCP), requesting intervention. The message can contain information such as a case number, key identifying details (patient name and ward) and extract of the patient's most recent observation data. Such information is in addition to the risk status level and request to respond.
2. As a message sent to a pager. The pager message is sent to the appropriate HCP. In addition to the risk level and request to respond, the message preferably contains a case number, key identifying details and an extract of the patient's most recent observation data.
3. As a message sent to a Handheld Wireless communications or computing device such as a PDA. If the HCP is within the confines of the hospital, the hospital has a wireless network, and the HCP has a PDA, an alert message is displayed on the PDA. The content of the message is the same as for the pager or SMS messages. The HCP can review the patient's data online from terminals connected to the administration system 105 and located around the hospital.
4. By automatic placement of a telephone call to the HCP's mobile or cell phone. The communication module automatically generates a voice message or provides an interactive voice response (VR) interface with the HCP in order to convey the relevant patient details.

A health care provider receiving an intervention request has two alternatives; he or she can:
1. Positively accept the intervention request alert by responding at step 945 before the acceptance period expires, via a cell or mobile phone (SMS), Interactive Voice Response system (IVR), or PDA or other device if they are within range of the hospital's wireless network. The system 100 then tags the health care provider as having accepted the request and he/she will be required to attend the patient within the corresponding response time; or
2. Reject the request either by not responding to the request within the specified acceptance time, or by positively rejecting the request via SMS, cell phone, PDA etc.

Health care personnel can respond at step 945 via the following mechanisms to a request for intervention:
1. SMS. The health care resource replies to the SMS message with a ‘Y’ (to indicate that the request is accepted) or ‘N’ (the intervention request is declined). The system 100 then updates the database to record the health care provider's acceptance or non-acceptance of the request.
2. IVR. Where a health care provider receives a request for intervention via a pager, he/she is required to call a toll free number, enter the case number (contained in the pager message), the HCP's hospital ID number and select either ‘accept’ (for example, in response to the voice message ‘press 1 on your phone to accept the intervention request’) or ‘reject’ (‘press 2 on your telephone to reject the intervention request’).

3. PDA or other wireless device. A health care provider within range of the hospital's wireless network can accept or reject an intervention request by sending a message through his/her PDA. Where the health care provider rejects the request he/she can, if he/she chooses to, assign the request to another health professional by selecting the relevant name from a drop down menu.

Depending on the risk status level, a health care provider that does not positively reject or accept an intervention request at step 945 may be contacted again at step 940 after a configurable time period has elapsed (as indicated by the “Recommended Maximum Time Between Cycles” column in the Table below). This continues until either the health care provider positively accepts or rejects the intervention request at step 950, or it is determined at step 975 that the corresponding response cycle limit (as indicated by the in the “Maximum Number of Communication Cycles” column in the Table below) has been reached, where the product of the cycle time period and the maximum number of cycles provides the acceptance period for the corresponding risk status level. When a response cycle limit is reached, the next higher risk level status is assigned to the patient at step 982, and hospital management is alerted at step 980. A new intervention/communications cycle begins with a higher alert status and consequent assignment of more senior personnel then commences at step 930 to expedite the provision of appropriate care to the patient.

In the event that the health care provider rejects the intervention request (as determined at step 950 ) within the prescribed cycle limits, the system then sends an intervention request to the next most appropriate health care provider at steps 930 to 940. As the cycle limit has not been exceeded, the risk status level is not escalated.

| | | | | | Maximum | Recommended | |
|---|---|---|---|---|---|---|---|
| | Intervention | | | | Number | Maximum Time | |
| | Response | | Acceptance | | of Communication | Between | |
| Status Level | Times | | Time Period | | Cycles | Cycles | |
| Status level - 0 | n/a | | n/a | | n/a | n/a | |
| Status level - 1 | 3-8 | hours | 180 | mins | 4 | 45 | mins |
| Status level - 2 | 1-3 | hours | 90 | mins | 3 | 30 | mins |
| Status level - 3 | 10-60 | mins | 24 | mins | 3 | 8 | mins |
| Status level - 4 | 0-10 | mins | 120 | sec | 2 | 60 | secs |
| Status level - 5 | 0-1 | mins | 10 | sec | 1 | 10 | secs |

A health care provider that accepts an intervention request will usually be required to attend the patient bedside (at step 960 ). The selected provider will have a defined bedside attendance response time standard to meet. For example, a provider that accepts a risk status Level 5 intervention request may have to meet a standard of attending the patient's bedside attendance within 2 minutes. Having accepted an intervention request alert at step 950, the health care provider will receive a reminder to attend the patient bedside shortly before the intervention response time has expired.

In the case where the primary healthcare provider attends the patient's bedside he or she is required to enter a unique identifier into the bedside PDA/tablet to positively confirm bedside attendance. The health care provider can enter his/her own details to log attendance, or alternatively another attending health care provider, such as a nurse, can do it on his/her behalf. When this occurs, all existing intervention requests for that specific patient are cancelled at step 967, and at step 969 an appropriate message is sent to any other health care providers who have been requested to attend the bedside. The attending health care provider administers treatment (at step 965 ), and the process of patient observation and risk assessment continues (at step 985 ).

If the health care provider does not meet the response time requirements at step 955, the communications module 120 treats the non-attendance as a positive rejection of the request for intervention. Hospital management is alerted at step 980, the risk status is incremented by one level (unless it is already at level 5 ) at step 982, and a higher level intervention activity is thereby requested and the communications module, 120 contacts the next most applicable health care resource at step 930, as described above.

If a patient's condition normalises after one or more health care providers have been requested to attend, any existing intervention requests are cancelled, and the responding provider notified of the cancellation in the same manner as the initial request.

Nurses within the ward in which an intervention request is activated receive an electronic full copy of the request and its status at step 940.

The human resource module 125 is a labour management tool. Available doctors for intervention requests are identified by checking check boxes displayed adjacent to their names on a personnel display page generated by the system 100. The communications module 120 then uses this information to determine which health care providers are on call/rostered and directs intervention requests to these resources.

The event logging and system analysis module 130 provides clinical and business metrics that assist in decision-making, process improvement and clinical governance. This module 130 analyses and evaluates the overall performance of doctors and nurses on an individual and event basis. For example, an adverse event (AE) can be analysed across almost any factors or potential contributory causes to provide forensic data and a deeper understanding of why a patient may have had an AE and the contributing factors attached thereto. Where events have occurred that require reconstruction and detailed information, the event logging and system analysis module 130 can assist to determine what happened. This module 130 can also help identify personnel that require additional training for performance or skill deficiencies. The event logging and system analysis module 130 performs the following activities:
(i) Collation, analysis and interpretation of patient and medical staff data;
(ii) Statistical comparisons and risk profiling to detect aberrant processes, responses and procedures;
(iii) Benchmarking and reporting; and
(iv) Forensic reconstruction of events, decisions and activities that occurred before, during and after an AE.

The module 130 also provides operational metrics that can be used to generate a business oriented report providing summary data for key performance measures such as average stay per patient, bed utilisation, AE incidence, mortality rates, and key human resource ratios.

In an alternative embodiment, the administration system includes or interfaces with an artificial intelligence (AI) engine (not shown) for assisting performance of the risk assessment module 115 in its decision making functions. For this purpose, the Al engine interfaces with the data repository 110, data storage 140 and risk assessment module 115 so as to more intelligently assign risk status levels according to received clinical observation data over a period of time. The output of the AI engine is also provided to the event logging and system analysis module 130 to provide further information to assist the hospital management in its decision making.

The AI engine provides guidance to hospital management on what benchmarks and process data values are recommended for use within the specific hospital environment. The Medical Committee of each hospital is then responsible for approving and updating the operating parameters within the system, including the risk assessment rules. The administration system 105 maintains a log of approvals and updates and access is restricted to only senior medical staff.

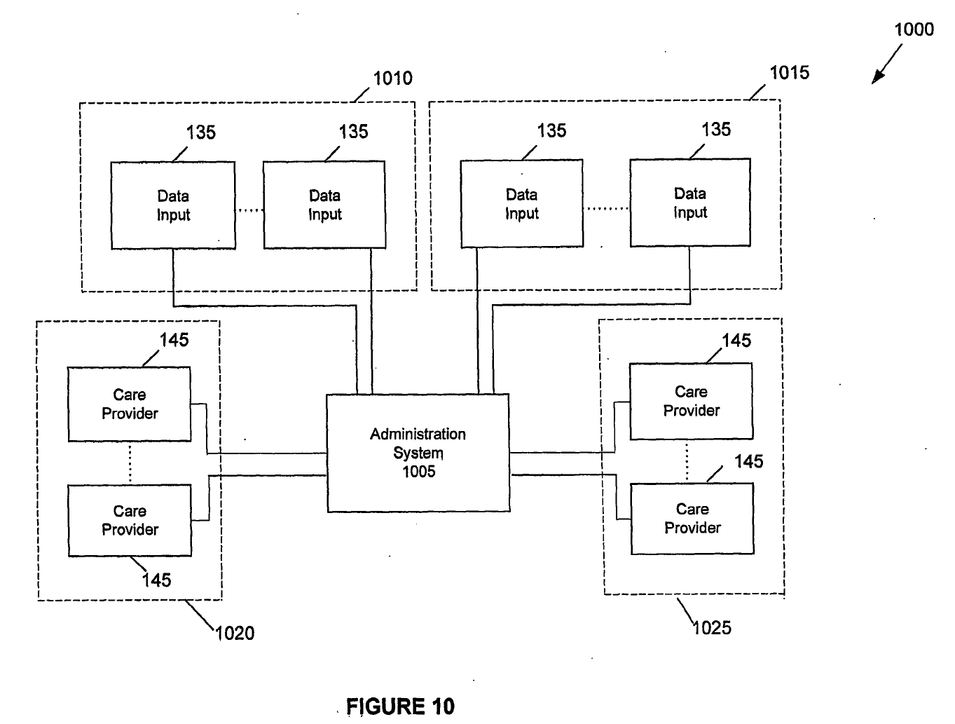

Although the administration system 1005 has been described as being located on the hospital premises, a further alternative embodiment may be more suitable for a hospital care network involving a common hospital management entity or a group of affiliated hospitals using centralised information technology infrastructure. This further alternative embodiment provides a health care system 1000 based on an application service provider (ASP) model 1005, as shown in FIG. 10. In this embodiment, the administration system 1005 is a centralised system in communication with numerous data capture devices 135 and health care providers 145, even though the administration system 1005 may be located quite remotely therefrom.

Patient clinical data is collected at the bedside and transmitted to the remote administration system 1005 for processing as described above in relation to FIGS. 1 to 9 and the first described embodiment. Where necessary, the communications module 120 of the administration system 1005 communicates with one or more health care providers 145 corresponding to the patients for which data was captured. For example, one hospital location 1010 may have a large number of data capture devices 135 for its patients and a corresponding group 1020 of health care personnel for providing health care to those patients. Simultaneously, another hospital location 1015 may provide clinical observation data from its capture devices 135 to the central administration system 105 in order for its patients to receive health care from a separate group 1025 of health care providers at that location.

In this way, groups of hospitals, having in the order of 20 or more wards or locations, may employ a central administration system such as administration system 1005 so as to save costs instead of establishing a separate infrastructure for each location. Thus the system 1000 is suitable for servicing -a large number of hospital locations or wards, but is also suitable to service a single hospital.

Accordingly, the health care systems described herein can be deployed in a wide variety of hospital infrastructure environments, ranging from a stand-alone personal computer (PC) environment in an individual ward, through to an integrated wireless network with electronic bedside tablet PCs delivering alerts through a communications gateway direct to health care professionals via PDAs, palmtop computers and/or mobile phones.

Advantageously, the health care systems are capable of extracting and receiving data from legacy Patient Administration Systems (‘PAS’) and core clinical systems including patient and administrative data—personal, administrative, clinical and other details. In addition, the health care systems can extract at least some of the clinical observation data from bedside monitoring devices and/or other diagnostic devices.

Advantageously, the system provides a high degree of user configurability to meet the needs of a diverse range of hospital,—specialist hospital, teaching hospitals, country hospital down to the ward level-i.e., General, Obstetrics, Paediatrics, etc.

Although the health care systems described above have been described above as receiving patient clinical data entered manually by health care personnel, it will be apparent that the systems could be adapted to automatically interrogate electronic patient monitoring devices equipped with suitable communications interfaces (e.g., RS-232, IEEE-488, GPID, USB, etc.) This would enable the health care systems to continually monitor the health parameters of a patient without any requirement for action on the part of any health care providers.

Moreover, although the health care process has been described above in terms of generating risk index values for each of five key health systems and then determining a risk status level on the basis of the risk index values, it will be apparent that the clinical data for each patient can be processed in a variety of ways to determine a measure of risk to the patients health.

Many modifications will be apparent to those skilled in the art without departing from the scope of the present invention as herein described with reference to the accompanying drawings.

1. A process executed by a computer system for facilitating the provision of health care to a patient, including the steps of:
receiving patient data relating to the health of a patient;
processing said patient data to determine a risk status providing an indication of risk to the patient's health;
selecting a health care provider to attend said patient on the basis of said risk status; and
transmitting a direction to said health care provider to attend the patient.

2. A process as claimed in claim 1, wherein said direction includes said risk status.

3. A process as claimed in claim 1, wherein said direction includes said risk status and at least part of said patient data.

4. A process as claimed in claim 1, wherein said step of transmitting includes transmitting successive directions to respective health care providers to attend the patient, whereby a direction is transmitted to a health care provider only if the one or more health care providers previously directed have not responded to their respective directions.

5. A process as claimed in claim 4, wherein a health care provider is considered to have not responded to a direction if a message indicating the health care provider's intention to attend the patient is not received within a first time period, or if a message confirming that the health care provider has attended the patient is not received within a second time period.

6. A process as claimed in claim 5, wherein said first time period and said second time period are determined by said risk status.

7. A process as claimed in claim 4, including determining at least one increased risk status for at least one of said successive directions.

8. A process as claimed in claim 7, wherein each direction includes a corresponding risk status.

9. A process as claimed in claim 1, wherein said patient data includes a plurality of health parameters of said patient.

10. A process as claimed in claim 9, wherein said risk status is determined on the basis of said plurality of health parameters and a do-not-resuscitate (DNR) status of said patient.

11. A process as claimed in claim 9, wherein said risk status is determined on the basis of said plurality of health parameters and one or more co-morbidity factors.

12. A process as claimed in claim 9, wherein said plurality of health parameters includes at least two of blood pressure, heart rate, respiration rate, oxygen saturation, consciousness level, urine output, temperature, level of consciousness, and pain score.

13. A process as claimed in claim 9, wherein said step of processing said patient data includes processing said plurality of health parameters to determine measures of risk, and determining said risk status on the basis of said measures of risk.

14. A process as claimed in claim 13, wherein said measures of risk correspond to respective health systems of said patient.

15. A process as claimed in claim 14, wherein said health systems of said patient include neurological, respiratory, cardiovascular, urinary, and temperature health systems.

16. A process as claimed in claim 13, wherein said risk status is selected from a plurality of predetermined risk status levels.

17. A process as claimed in claim 16, wherein said measures of risk are selected from a plurality of predetermined risk levels.

18. A process as claimed in claim 17, wherein said determining includes:
if one or more of said measures of risk is equal to the highest of said plurality of predetermined risk levels, then selecting said risk status as the highest of said plurality of predetermined risk status levels; and
otherwise, if two or more of said measures of risk are greater than the lowest of said plurality of predetermined risk levels, then selecting said risk status as the highest of said two or more measures of risk, and incrementing said risk status by one level unless said risk status is equal to the highest of said plurality of predetermined risk levels.

19. A process as claimed in claim 13, wherein said risk status is determined on the basis of first rules applied to said measures of risk.

20. A process as claimed in claim 19, wherein the measures of risk are determined on the basis of second rules applied to at least some of said health parameters.

21. A process as claimed in claim 19, wherein said first rules and said second rules are configurable by a user.

22. A process as claimed in claim 18, wherein said determining further includes incrementing said risk status by one level if a selected health care provider has not responded to said direction.

23. A process as claimed in claim 22, wherein said determining further includes limiting the level of said risk status to less than the highest of said plurality of predetermined risk levels unless the patient is experiencing a life-threatening event.

24. A process as claimed in claim 22, wherein said determining further includes limiting the level of said risk status to less than the highest of said plurality of predetermined risk levels if the patient is subject to a not-for-resuscitation order, even if the patient is experiencing a life-threatening event.

25. A process as claimed in claim 1, wherein the direction is transmitted to one or more wireless devices of said health care provider.

26. A process as claimed in claim 1, wherein the direction is transmitted to a first device associated with said health care provider, and the process includes transmitting said direction to a second device associated with said health care provider if said health care provider does not reply to said direction.

27. A process as claimed in claim 1, wherein the direction is transmitted to at least two devices associated with said health care provider at the same time if said risk status is indicative of a significant health risk to said patient.

28. A process as claimed in claim 25, wherein said one or more wireless devices includes one or more of a telephone, a personal data assistant, and a portable computing device.

29. A process as claimed in claim 1, including receiving availability data indicating the availability of at least one health care provider, wherein a health care provider is selected only if said health care provider is available to attend said patient.

30. A process as claimed in claim 1, wherein said step of selecting includes selecting a type of health care provider on the basis of said risk status.

31. A process as claimed in claim 30, wherein the type of health care provider includes one of a nurse, a doctor, a registrar, a consultant, and a cardiac arrest response team.

32. A process as claimed in claim 31, wherein said step of selecting includes selecting a health care provider of the selected type on the basis of availability data indicating the availability of the health care provider to attend said patient.

33. A process as claimed in claim 1, wherein the direction transmitted to said health care provider includes an intervention activity associated with said risk status.

34. A process executed by a computer system for facilitating the provision of health care to a patient, including the steps of:
receiving patient data relating to the health, of said patient;
determining a risk status of said patient based on said patient data;
transmitting a first direction to a first health care provider to attend the patient, the first direction including the risk status of the patient;
determining whether the first health care provider confirms attendance at the patient; and
transmitting a second direction to a second health care provider to attend the patient if attendance by the first health care provider was not confirmed.

35. A process as claimed in claim 34, wherein the second direction includes an increased risk status of the patient.

36. A process as claimed in claim 35, wherein the second direction includes a second time period for attending the patient.

37. A process as claimed in claim 36, wherein the first time period is associated with the determined risk status, and the second time is associated with the increased risk status.

38. A process as claimed in claim 36, wherein the second time period is equal to or less than the first time period.

39. A process as claimed in claim 36, wherein the process further includes the steps of:
determining whether the health care provider confirms attendance at the patient within the second period; and
transmitting a third direction to a third health care provider to attend the patient if attendance by the second health care provider was not confirmed within the second time period.

40. A process as claimed in claim 39, wherein the third direction includes a further increased risk status of the patient.

41. A process as claimed in claim 39, wherein the third direction includes a third time period for attending the patient, the third time period being equal to or less than the second time period.

42. A process as claimed in claim 39, wherein the third time period is less than the first time period.

43. A patient care process executed by a computer system, including the steps of:
determining a risk level of a patient; and
repeatedly requesting one or more medical or health care providers to attend the patient if the risk level is above a predetermined level and the patient is unattended by said health care providers.

44. A health care system having components for executing the steps of any one of claims 1 to 43.

45. A computer readable storage medium having stored thereon program code for executing the steps of any one of claims 1 to 43.

46. A system for facilitating the provision of health care to one or more patients, including:
computerised means for logging patient data relating to health of said one or more patients;
an administration system in communication with said computerised means and configured to determine a risk status of each of said one or more patients based on the patient data, said administration system being further configured to, for each patient: transmit a first direction to a first health care provider to attend the patient, depending on the risk status of the patient; determine whether the first health care provider has confirmed attendance at the patient within a first time period; and transmit a second direction to a second health care provider to attend the patient within a second time period if attendance by the first health care provider was not confirmed.

47. A system as claimed in claim 46, wherein the second time period is equal to or less than the first time period.

48. A system as claimed in claim 46, wherein the first and second directions are effected by automatic transmission of a message to portable electronic devices associated with the respective first or second health care providers.

49. A system as claimed in claim 48, wherein the first and second directions are transmitted as wireless communications.

50. A system as claimed in claim 46, wherein the patient data includes data relating to a plurality of health parameters.

51. A system as claimed in claim 46, wherein the first direction is only transmitted when the risk status is equal to or above a threshold level.

52. A system as claimed in claim 46, wherein the first and second directions include information concerning the risk status of the patient.

53. A system as claimed in claim 46, wherein the first and second directions include a request to confirm that the relevant health care provider intends to comply with the direction.

54. A system as claimed in claim 46, wherein the administration system increases the risk status of the patient if it determines that the first health care provider has not confirmed attendance at the patient within the first time period.

55. A system as claimed in claim 46, wherein the administration system is further configured to determine whether the second health care provider has confirmed attendance at the patient within the second time period and to transmit a third direction to a third health care provider to attend the patient within a third time period if attendance by the second health care provider was not confirmed within the second time period.

56. A system as claimed in claim 55, wherein the third time period is equal to or less than the second time period.

57. A system as claimed in claim 46, wherein the computerised means include a plurality of computerised devices networked with, but located remotely from, the administration system.

58. A system as claimed in claim 46, wherein each computerised communication device is located nearby the one or more patients.

59. A system as claimed in claim 46, wherein the computerised device is a wireless handheld device.

60. A system as claimed in claim 46, wherein the computerised device includes a personal computer with appropriate input means for logging the patient data.

61. A system as claimed in claim 46, wherein the administration system includes a centralised server having a risk assessment module for determining the risk status and a communications module for transmitting directions to health care providers.

62. A system as claimed in claim 46, wherein directions to the health care provider are transmitted to at least two contact devices of the health care provider.

63. A system as claimed in claim 62, wherein a direction to the health care provider is transmitted to at least two contact devices of the health care provider at the same time.

64. A system as claimed in claim 46, wherein the direction is in the form of a recorded voice message directed to a telephone number associated with the health care provider.

65. A patient care system including:
at least one electronic device for recording patient data relating to the health of one or more patients;
a central server in communication with the at least one electronic device and configured to repeatedly contact one or more health care providers to request attendance at least one of the one or more patients if the patient data indicates that the health of the at least one patient is above a predetermined risk level and the at least one patient is unattended.

66. A patient care system, comprising:
means for determining a risk level of a patient; and
means for repeatedly contacting one or more medical or health care personnel to attend the patient if the risk level is above a predetermined level and the patient is unattended.
